(19) United States
(12) Patent Application Publication
(10) Pub. No.: US 2010/0216174 A1
(43) Pub. Date: Aug. 26, 2010

(54) METHOD OF EVALUATING THROMBOGENIC MICROPARTICLES

(76) Inventors: Bernd Binder, Wien (AT); Helga Vetr, Moedling (AT); Hirotaka Isobe, Vienna (AT)

Correspondence Address: KF ROSS PC, 5683 RIVERDALE AVENUE, SUITE 203 BOX 900, BRONX, NY, 10471-0900, US

(21) Appl. No.: 12/667,294

(22) PCT Filed: Jul. 4, 2008
(86) PCT No.: PCT/AT08/00244

§ 371 Date: Mar. 12, 2010

(30) Foreign Application Priority Data
Jul. 6, 2007 (AT) A 1046/2007

(51) Int. Cl. C12Q 1/56

(52) U.S. Cl. 435/13

(57) ABSTRACT

A method and a kit are disclosed for evaluating thrombogenic microparticles (MP). The principle is based on the evaluation of thrombin generation in platelet poor plasma (PPP) and in plasma made microparticle free (MPFP) by centrifugation or filtration. The difference in thrombin generation between PPP and MPFP correlates with number and activity of thrombogenic microparticles. Evaluating thrombin generation of different standardized amounts of MPs in standard MPFP allows to calculate the number of thrombogenic MP from the difference in thrombin generation between PPP and MPFP.

The invention relates to a method to determine number and functional activity of thrombogenic microparticles in plasma according to the classifying part of claim 1.

### BACKGROUND OF THE INVENTION

Role of Microparticles in Disease

During the last years there was increasing evidence that cell-derived microparticles in the blood stream are an important marker for thrombosis as well as for other pathological states such as infection and/or inflammation (reviewed in reference list).

Microparticles (MP) are vesicles (membranous fragments) that can be released into the blood stream by all types of circulating blood cells as well as by endothelial cells. The size of MPs is less than 1 μm. Their surface is composed of phospholipids, antigenic markers of their parent cells, and proteins such as tissue factor, which together with phospholipids is promoting coagulation. The release of microparticles can be stimulated by various activation processes such as complement lysis, oxidative stress, high shear stress. Due to their different origins and release upon various stimuli, MPs are very heterogeneous in size and composition as shown by George et al 6.

Methods of Evaluating of Microparticles

Analysis of microparticles currently involves either flow cytometric analysis or ELISA (enzyme linked immuno sorbent assay) based systems.

Flow cytometric analysis can be used to analyse the number of microparticles in plasma samples by evaluating the number of particles between 0.1 and 1 mm in size using standardized size markers ⁷. Alternatively, specific features of MPs can be used for their evaluation in FACS analysis or by use of ELISA based systems such as their property to expose negatively charged phospholipids to which Annexin V is bound. Citation In addition or separate from Annexin V, antibodies directed against surface markers on MPs can be used to determine their cellular origin.

These surface markers are derived from their parent cells such as endothelial, neutrophil, monocyte, or erythrocytes citations. In addition, non-cell specific markers such as tissue factor as a determinating factor for the thrombogenic activity of MPs activity can be used for characterization of Mps. ^{7,8}

Limitation of existing methods of evaluating Microparticles All the above described methods analyse structures or molecules on the surface of MPs but not the functionality of the MPs. Moreover, they are limited to analysis of subpopulation of MPs. Not only do the cell specific markers only detect subpopulation of MPs but also for Annexin V it has been shown that only a fraction of MPs is positive for Annexin V binding (⁹).

Common to all methods is the limitation by sample preparation. Usually MPs are prepared by centrifugation. High speed centrifugation never can remove 100% of microparticles because some of them have the same density as plasma ¹⁰. Furthermore, collecting MPs by centrifugation is not easy to be standardized, and in most cases not quantitative. The alternative of density gradient centrifugation is suited only for research labs, needs expensive equipment and is time and labour intensive.

Regarding feasibility of these assays it should be noted that flow cytometry needs specialized and expensive equipment and is usually not available for a routine clinical lab.

### AIM OF THE PRESENT INVENTION

As outlined above none of the existing assays has the potential to enter the clinical routine lab. All these approaches are hampered by either being dependent on expensive equipment or being laborious or by evaluating only a fraction of all thrombogenic MPs. Moreover, standardization of these assays is not easy to achieve.

It is therefore desirable to determine number and activity of circulating MPs by a standardized method. The aim of the present invention is to develop a method which allows rapid, reliable and is reproducible evaluation of thrombogenic MPs.

### SUMMARY OF THE INVENTION

Therefore object of the present invention is a method to determine number and functional activity of thrombogenic microparticles in plasma characterized by evaluating thrombin generation in PPP and MPFP prepared from the same sample, calculating the difference between these two values and relating such difference to the thrombogenic activity of a microparticle standard.

In another embodiment of the invention a test kit is presented including the tools and protocol for sample preparation as well as the reagents necessary for measuring thrombin generation. Moreover, standards and controls for quantification of the number of thrombogenic microparticles are included.

In another embodiment the option for automated analysis of samples is described.

This includes collection of filtered samples in sample containers which are compatible with an automated coagulation analyzer of the type CEVERON alpha with a “TGA Modul”

### BRIEF DESCRIPTION OF THE FIGURES

FIG. 1 is a calibration curve MPs (×100)−nM Thrombin (peak thrombin), On the abscissa is indicated the number of microparticles per μl (cts MP/μl) and on the ordinate nM peak thrombin as difference of thrombin generation between microparticles (MP) containing (MP added) and MP free plasma.

FIG. 2 shows the differences in peak thrombin between PPP and MFP. From the differences in peak thrombin between PPP and MPF shown in FIG. 2 the number of microparticles can be calculated. Control (Control Induviduals); MI ASA (Patient groups myocardial infarction treated with ASA); MI none (Patient groups myocardial infarction).

### EXAMPLES

Evaluation of the thrombin generating activity (TGA) of standard samples:

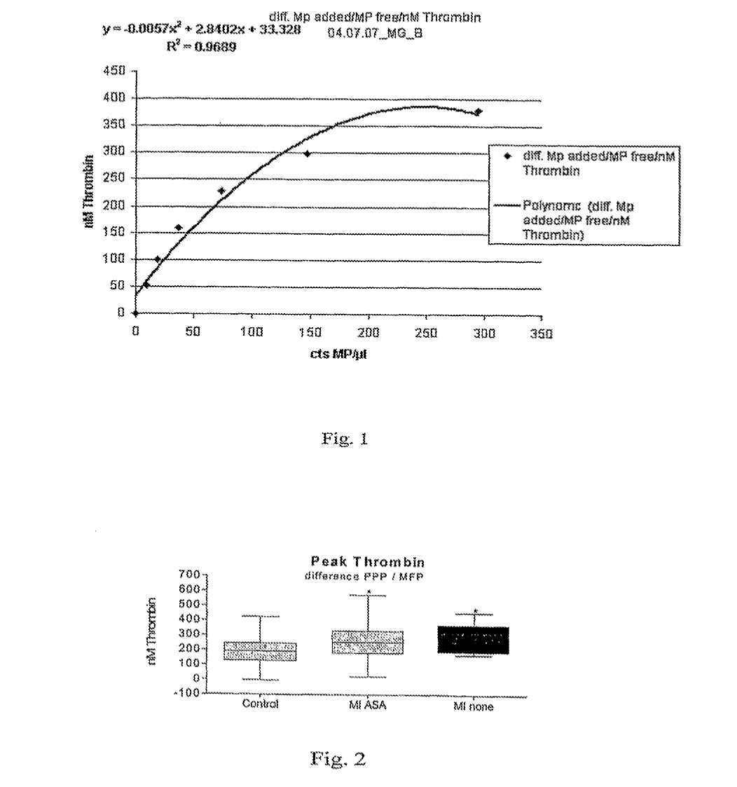

When different concentrations of microparticles are added to standard microparticle free plasma and thrombin generations measured a standard curve is obtained as shown in FIG. 1

Preparation of samples and evaluation of thrombin generation Platelet poor plasma is prepared from anticoagulated blood by centrifugation according to DIN 58905 (15 min at a minimum of 2500 g). Microparticle free plasma was prepared by filtration using a low-protein binding membrane with pore size 0.2 μm. (e.g. PALL, BioInertÒ). The preferred filtration units are AcroPrepÒ 96 well Filter plates (PALL 5042) with a Bio-InertÒ Membrane (hydrophilic nylon). Other filter plates with similar properties can be used. Alternatively, centrifugation can be used.

Thrombin generation in PPP and MPFP of each sample is measured. The difference in thrombin generation between PPP and MPFP is calculated and is related to the number of MPs present in the sample. As readout parameters from the thrombin generation curve peak thrombin or area under the curve can be used. By comparison with the standard curve (FIG. 1) the concentration of thrombogenic MPs in the sample can be determined. In FIG. 2 mean values for TGA in control samples and in samples from different groups of patients are shown for platelet poor plasma and for microparticle free plasma.

Calculation of the number of thrombogenic microparticles: The differences in thrombin generating activity (e.g. peak thrombin) between platelet poor plasma and microparyticle free plasma are calculated and the value is read from the calibration curve:

| TGA in PPP nM | TGA in MPFP nM | Difference nM | |
|---|---|---|---|
| thrombin | thrombin | thrombin | Microparticle/ml |
| 125 | 27 | 98 | 2.200 |
| 387 | 75 | 312 | 15.000 |
| 258 | 43 | 215 | 7.500 |
| 175 | 44 | 131 | 4.000 |

The disclosure and content of the following references is included in this application by reference.

### REFERENCE LIST

1. VanWijk, M. J., VanBavel, E., Sturk, A. & Nieuwland, R. Microparticles in cardiovascular diseases. Cardiovasc. Res. 59, 277-287 (2003).
2. Freyssinet, J. M. Cellular microparticles: what are they bad or good for? J. Thromb. Haemost. 1, 1655-1662 (2003).
3. Horstman, L. L., Jy, W., Jimenez, J. J. & Ahn, Y. S. Endothelial microparticles as markers of endothelial dysfunction. Front Biosci. 9, 1118-1135 (2004).
4. Horstman, L. L., Jy, W., Jimenez, J. J., Bidot, C. & Ahn, Y. S. New horizons in the analysis of circulating cell-derived microparticles. Keio J. Med. 53, 210-230 (2004).
5. Morel, O. et al. Procoagulant microparticles: disrupting the vascular homeostasis equation? Arterioscler. Thromb. Vasc. Biol. 26, 2594-2604 (2006).
6. George, J. N., Thoi, L. L., McManus, L. M. & Reimann, T. A. Isolation of human platelet membrane microparticles from plasma and serum. Blood 60, 834-840 (1982).
7. Jy, W. et al. Measuring circulating cell-derived microparticles. J. Thromb. Haemost. 2, 1842-1843 (2004).
8. Horstman, L. L. & Ahn, Y. S. Platelet microparticles: a wide-angle perspective. Crit Rev. Oncol. Hematol. 30, 111-142 (1999).
9. Horstman, L. L., Jy, W., Jimenez, J. J. & Ahn, Y. S. Endothelial microparticles as markers of endothelial dysfunction. Front Biosci. 9, 1118-1135 (2004).
10. Horstman, L. L., Jy, W., Jimenez, J. J., Bidot, C. & Ahn, Y. S. New horizons in the analysis of circulating cell-derived microparticles. Keio J. Med. 53, 210-230 (2004).

1. A method of determining number and functional activity of thrombogenic microparticles in plasma characterized by evaluating thrombin generation in PPP and MPFP prepared from the same sample, calculating the difference between these two values and relating such difference to the thrombogenic activity of a microparticle standard.

2. The method as in claim 1 wherein thrombin generation is determined by a thrombin generation assay using a fluorigenic substrate and monitoring cleavage of the substrate over time.

3. The method according to claim 2 wherein peak thrombin is used as parameter for evaluating microparticles.

4. The method according to claim 2 wherein the area under the curve is used as parameter for evaluating microparticles.

5. The method according to claim 1 wherein MPFP is prepared by centrifugation.

6. The method according to claim 1 wherein MPFP is generated by filtration.

7. The method according to claim 1 wherein a microparticle standard with MPs derived from the plasma of blood donors is used.

8. The method according to claim 1 wherein a microparticle standard with MPs derived from cells is used.

9. The method according to claim 8 wherein the cellular source of microparticles are leukocytes.

10. The method according to claim 8 wherein the cellular source of microparticles are endothelial cells.

11. The method according to claim 8 wherein the cellular source of microparticles are red blood cells.

12. The method according to claim 8 wherein the cellular source of microparticles are platelets.

13. The method according to claim 6 wherein the MP standard is suspended in a standardized MP free plasma.

14. The method according to claim 6 wherein the MP standard is suspended in MPFP of the patient.
